(19) United States
(12) Patent Application Publication
(10) Pub. No.: US 2010/0004703 A1
(43) Pub. Date: Jan. 7, 2010

(54) Energy Medicine Treatment Methods

(76) Inventor: M. Anthony Smith, Post Falls, ID (US)

Correspondence Address: WELLS ST. JOHN P.S., 601 W. FIRST AVENUE, SUITE 1300, SPOKANE, WA, 99201, US

(21) Appl. No.: 12/407,446

(22) Filed: Mar. 19, 2009

Related U.S. Application Data
(63) Continuation-in-part of application No. 11/544,905, filed on Oct. 5, 2006, now abandoned.
(60) Provisional application No. 60/802,980, filed on May 23, 2006.

(51) Int. Cl. A61N 1/00; A61N 2/00; A61B 5/00

(52) U.S. Cl. 607/2; 600/9; 600/300

(57) ABSTRACT

An energy medicine treatment method includes ascertaining a health dysfunction of a patient, the patient exhibiting a head point(s) on a surface of the cranium and the head point(s) being biologically active in a manner conducive to treatment of the dysfunction. Without identifying a specific location of the head point(s), the method includes stimulating a sufficiently large portion of the cranium to clear the head point(s) and to enable the patient's body to improve proper function of the dysfunction.

### RELATED PATENT DATA

This patent is a continuation-in-part of U.S. patent application Ser. No. 11/544,905, filed Oct. 5, 2006, which claims the benefit of priority under 35 U.S.C. §119 to U.S. Provisional Application No. 60/802,980, filed May 23, 2006, each of which are incorporated herein by reference.

### BACKGROUND OF THE INVENTION

Energy medicine represents an area of increasing interest. In one sense, the concept of energy medicine relies upon energy flux through the body and its interactions with the brain to ascertain the existence of a health dysfunction and to prompt the brain to remedy the dysfunction. Muscle response testing (MRT) is a widely used technique based upon the principles involved in energy medicine. Kinesiology, applied kinesiology, and other terms are sometimes used to refer to muscle response testing or to similar techniques. Within the context of the present document, “muscle response testing” refers to the practice of determining whether or not a muscle may maintain a particular position with a minimal amount of pressure applied to the muscle during application of a stimulus. Weakening of the muscle indicates a positive finding.

One principle of energy medicine involves the concept that the brain may be considered an electrical generator, sending electrical impulses to every cell in the body and generating an electromagnetic field. Some parts of the body have more electrical charge or a different polarity compared to others. For example, the fingertips, palm, and area around the mouth and umbilicus (also known as the navel) have more electrical charge. The palm has a positive charge and the back of the hand has a negative charge.

In the case of allergies, the AllerTouch® method practiced and taught previously by the present inventor, M. Anthony Smith, D.C. of Dynamic Health in Coeur d'Alene, Id., uses MRT to ascertain a patient's allergy. With the patient either sitting or standing, his arm is raised perpendicular to his body and straight at the elbow and wrist, the hand open and relaxed, and the palm facing downward. Any metal jewelry around the neck, wrists, or ankles is removed prior to testing. An allergen test kit may be used containing glass vials, such as the test kits available from Allergies Lifestyle & Health (ALH) located in Eatonville, Wash. The ALH vials contain water radionically imprinted with the allergen or a material containing the allergen. The radionic imprinting is done according the knowledge of those of ordinary skill in the field of energy medicine.

The doctor's open hand is placed comfortably against the top of the patient's raised wrist, applying light downward pressure while the patient lightly touches the center of a vial with the tip of his index finger on the other hand held at about a 45° angle to the vial for about 2 seconds. Weakening of the patient's arm (deltoid muscle) indicates the presence of an allergy corresponding to the vial's contents. Since the index fingertip is very electrical, when the patient touches the glass test vial, the body's energy easily passes through the glass and returns information to the brain about what is in the vial. Metal test vials prevent such a process. The doctor partially removes from the kit any vials producing a positive finding (presence of the allergy) so that each positive finding may be easily confirmed later.

After testing all allergens in the kit, positive findings may be confirmed by performing MRT with one of the patient's arms while the patient gently squeezes the corresponding vial in the palm of his other hand. The doctor may optionally ascertain the location of a particular allergy, i.e. the affected organ. The patient holds a vial that tested positive in the palm of his hand while the doctor performs MRT as the doctor touches each organ point shown in FIG. 11 with the tip of his index finger. Alternatively, the doctor may perform MRT while the doctor holds a vial over each organ point shown in FIG. 11. A positive finding indicates the location of the allergy.

In the AllerTouch® method, treatment involves first identifying on the patient's cranium the location of “head points” corresponding to the allergy. A “head point” is a small area on the patient's cranium that becomes evident with MRT when the doctor or patient touches the allergy point shown in the FIG. 12 chart of health stressors. The allergy point becomes tender and exhibits a very much localized electromagnetic charge when the patient has an allergy. Touching the allergy point essentially causes a brief short circuit in the nervous system and muscles in the body become temporarily weak. Head points also exhibit a very much localized electromagnetic charge and are often tender to the touch.

Locating a head point involves the doctor performing MRT while the patient holds the vial and touches the allergy point with the fingertip of the same hand. The doctor scans the patient's cranium during MRT by touching with the fingertips of one hand until a head point produces a positive finding. Next, the doctor identifies which fingertip triggered the head point by performing MRT for each fingertip. Responsiveness of the head point will mentally reset within the patient to allow identifying which fingertip initially triggered the head point.

Treatment involves the patient gently squeezing the appropriate vial in the palm of one hand while touching the organ point of the affected organ, if ascertained, with the fingertip(s) of the other hand. The doctor lightly touches the tip of one index finger on the specific head point while lightly tapping with the thumb and middle finger of the other hand, first on the left and then on the right cerebral hemisphere, on top of the patient's head. The tapping “clears” the head point. Usually, there are multiple head points and the doctor must find and clear each one. The objective of the tapping includes prompting the brain to communicate to the body that it should no longer respond to the substance in the vial as an allergen. Effectiveness of treatment may be verified by performing MRT again while the patient gently squeezes the vial in the palm of one hand and then performing MRT yet again with the vial in the other hand. If muscle weakness occurs with the vial in either hand, then treatment may be repeated.

AllerTouch® may be adapted for treating bacterial, viral, or parasitic infections. The doctor performs MRT while the patient touches a test vial with the surface of the index fingernail or touches a stressor point of a bacterial, viral, or parasitic infection, as shown in FIG. 12. For a positive finding, the infection may be located when the patient touches the stressor point while the doctor performs MRT and individually touches organ points shown in FIG. 11 with the tip of his index finger.

For treatment, the doctor identifies the location of the specific head points by performing MRT while the patient touches the respective bacterial, viral, or parasitic stressor point. Next, the patient touches the stressor point while the doctor clears the head point as indicated above. The objective of the tapping includes prompting the brain to communicate to the body that it should no longer respond to the substance in the vial as benign and instead focus on addressing the infection.

Even though the AllerTouch® method has proven effective, locating and clearing head points can be a somewhat time consuming aspect of the overall patient treatment when considering all of the allergies, bacteria, viruses, and parasites potentially evaluated. Further, locating a head point, potentially multiple head points, adds a level of complexity to AllerTouch® for students of the technique.

### BRIEF DESCRIPTION OF THE DRAWINGS

Preferred embodiments of the invention are described below with reference to the following accompanying drawings.

FIG. 1 is a flow chart of initial procedures associated with an energy medicine testing method.

FIG. 2 is a flow chart of an energy medicine testing method for bacterial, viral, fungal, or parasitic infection.

FIG. 3 is a flow chart of an energy medicine testing method for allergies or elevated metals or chemicals.

FIG. 4 is a flow chart of an energy medicine testing method for a digestive HCl deficiency or a digestive enzyme deficiency.

FIG. 5 is a flow chart of an energy medicine testing method for a scar/organ nerve energy disruption.

FIG. 6 is a flow chart of an energy medicine testing method for a dysbiotic root canal.

FIG. 7 is a flow chart of an energy medicine testing method for an organ dysfunction.

FIG. 8 is a flow chart of an energy medicine testing method for muscle dysfunction.

FIG. 9 is a flow chart of an energy medicine testing method for vertebral subluxation.

FIG. 10 is a flow chart of an energy medicine testing method for a posterior or postero-lateral spinal disc.

FIG. 11 is an energy medicine chart of organ points.

FIG. 12 is an energy medicine chart of health stressors.

### DETAILED DESCRIPTION OF THE PREFERRED EMBODIMENTS

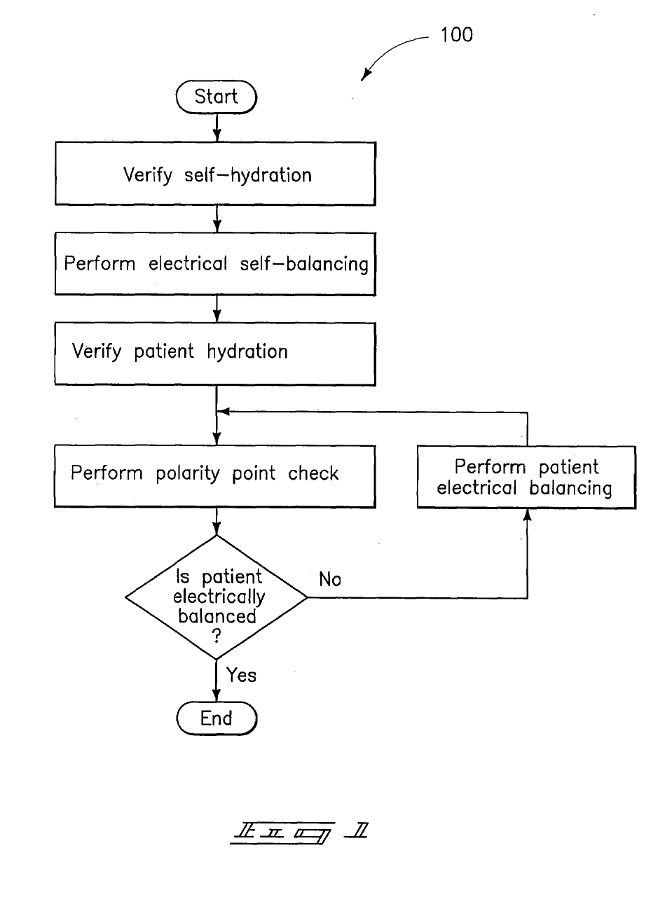

FIG. 1 shows a flow chart of initial preparations that may be used with aspects of the invention described herein. Method 100 in FIG. 1 helps ensure the accuracy of muscle response testing (MRT) and/or increase the effectiveness of subsequent treatments. Prior to beginning method 100, a patient's and a doctor's hands may be sterilized. Method 100 includes the doctor verifying adequate self-hydration. Since dehydration may affect MRT test results, the doctor may endeavor to drink an adequate amount of water while working.

As the term is used herein, “doctor” refers to a person skilled or specializing in healing arts, regardless of whether they are engaged in a profession that requires licensing to practice the profession. The skill or specialization of the doctor may arise solely from training in or study of the methods described herein. Consequently, the described methods are not limited to performance by professionally licensed individuals. Even so, treatment effectiveness might be enhanced with additional training in or study of other health related fields, such as traditional medicine, osteopathy, chiropractic, homeopathy, allopathy, naturopathy, etc.

Method 100 also includes the doctor performing electrical self-balancing. Electrical balancing includes the doctor placing the palm of one hand over the umbilicus (also known as the navel) and, with the thumb and index finger of the other hand, simultaneously rubbing underneath the head of each clavicle for 15 seconds. As an additional measure, the doctor may keep the palm of one hand over the umbilicus and, using the thumb and index finger of the other hand, simultaneously stroke lightly above the upper lip and below the lower lip toward the mouth. A possibility exists that electrical imbalance in the doctor will produce inaccurate MRT.

Method 100 next includes verifying patient hydration by having the patient drink a glass of water before MRT begins. The doctor next performs a polarity point check on the patient by performing MRT while touching the pad of the doctor's index finger on the highest part of the patient's nose bridge. Such location is known as the polarity point. The patient's arm (deltoid muscle) should weaken when the doctor touches the polarity point. If not, the patient might not be electrically balanced and electrical balancing as described above for the doctor may be performed by the patient. Method 100 includes repeating the polarity point check after electrical balancing. Accurate MRT of the patient might not be possible if the patient's arm does not weaken when the doctor touches the patient's polarity point with the pad of his index finger. A possibility exists that a patient may become electrically imbalanced during testing or subsequent treatments. Therefore, prudence warrants occasionally checking the polarity point.

After initial preparations of the doctor and patient, an energy medicine treatment method according to one aspect of the invention includes ascertaining a health dysfunction of a patient, the patient exhibiting a head point(s) on a surface of the cranium and the head point(s) being biologically active in a manner conducive to treatment of the dysfunction. Without identifying a specific location of the head point(s), the method includes stimulating a sufficiently large portion of the cranium to clear the head point(s) and to enable the patient's body to improve proper function of the dysfunction.

Advantageously, a wide variety of health dysfunctions have been identified as amenable to treatment by the described method. Even though the AllerTouch® method described in the Background of the Invention section was previously only used for allergies and bacterial, viral, and parasitic infections, aspects of the invention have found broader applicability to other health dysfunctions. The broader applicability may be facilitated by the simplified treatment. Notably, aspects of the invention include stimulating a sufficiently large portion of the cranium to clear the head point(s) without identifying a specific location of the head point.

Such treatment may be contrasted with AllerTouch®, which involved identifying the specific location of every head point that corresponded to an allergy or infection. However, the objective of the stimulation is similar to the objective of the tapping in AllerTouch®, namely, prompting the brain to communicate to the body to focus on addressing the health dysfunction. Consequently, the cranial stimulation could instead be referred to as energizing or stimulating the central nervous system, especially the brain. A variety of known acupressure or acupuncture points exist on the cranium. Although the stimulation technique differs and the aspects of the invention are not limited to using acupressure or acupuncture points, the possibility exists that effectiveness of the aspects of the invention and acupressure/acupuncture may be related by common phenomena. Even so, explanations supporting effectiveness are not limited to this theory.

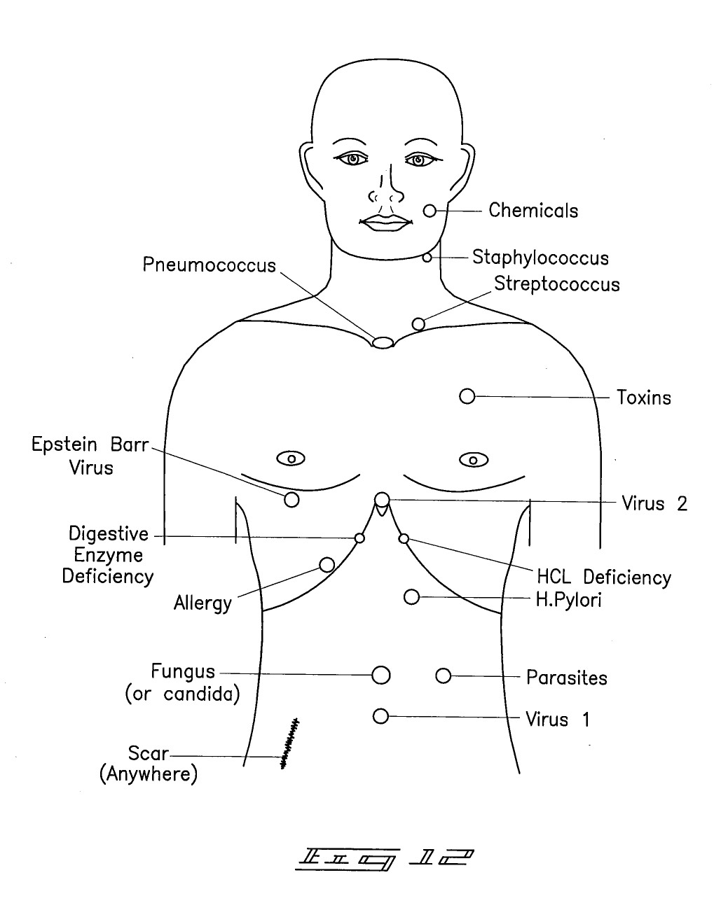

By way of example, the patient's body may exhibit a health stressor point, such as shown in FIG. 12, corresponding to the health dysfunction. Clearing the head point(s) may include touching the stressor point while stimulating the cranium. The treatment method may further include ascertaining an organ with which the health dysfunction is associated by using MRT and an organ point, such as in FIG. 11, exhibited by the patient's body. Clearing the head point may include touching the organ point while touching the stressor point and stimulating the cranium. In the context of the present document, “organ” refers to a differentiated part of an organism that performs a specific function. Accordingly, muscles, skin, bone, cartilage, etc. are considered organs in addition to typical “organs,” such as heart, lung, liver, etc.

Also by way of example, where the patient's body exhibits an organ point and the method further includes ascertaining an organ with which the health dysfunction is associated by using MRT, clearing the head point(s) may include touching the organ point while stimulating the cranium. That is, the stressor point is not necessarily used in clearing the head point(s). Also, test vials may be useful in treating certain health dysfunctions, as an alternative to using a stressor point. Accordingly, the MRT ascertaining the health dysfunction and the MRT ascertaining the associated organ may each use a test vial. Clearing the head point(s) may thus include touching the organ point while holding the test vial and stimulating the cranium. As one option, touching the organ point while holding the test vial may include holding the test vial against the organ point.

As stated in the Background of the Invention section, test vials described for the AllerTouch® method contained radionically imprinted water. However, generally, an actual substance that was imprinted on the water may be used instead of the imprinted water. The actual substance may be in a vial or touched directly. This approach might be problematic for some substances, such as bacteria, viruses, and parasites, but might work just as well for other substances, such as the sources of allergens. Consequently, references herein to using a “vial” should be considered to refer to using the actual substance, whether or not inside a container, such as a vial, or to using something representing the substance, such as radionically imprinted water.

Organ and/or stressor points may also be used for ascertaining the patient's health dysfunction. When the patient's body exhibits a stressor point, ascertaining the health dysfunction may include touching the stressor point during MRT. The method may further include ascertaining the organ with which the health dysfunction is associated by touching the organ point and touching the stressor point during MRT. However, as an alternative, ascertaining the health dysfunction may include touching the organ point during MRT without requiring touching a stressor point, if any. After ascertaining the existence of some general health dysfunction by such technique, the method may further include ascertaining a specific health dysfunction associated with the organ by touching the organ point while touching a stressor point during MRT.

Ascertaining the health dysfunction may include touching a test vial during MRT. In such case, the method may further include ascertaining the organ associated with the health dysfunction by using MRT while touching the organ point and the test vial. Touching the organ point while touching the test vial may include holding the test vial against the organ point.

Potential health dysfunctions addressed by aspects of the invention include, among other dysfunctions, those selected from the group consisting of a fungal infection, a fungal infection in a sinus, a Candida infection, elevated metals or chemicals, a digestive enzyme deficiency, a digestive HCl deficiency, a scar/organ nerve energy disruption, a dysbiotic root canal, muscle dysfunction, vertebral subluxation, and a posterior or postero-lateral spinal disc. None of the specifically listed dysfunctions are known to have been previously addressed by using muscle response testing and clearing a biologically active head point(s).

In some cases, a health symptom exhibited by the patient may be caused by the health dysfunction and clearing the head point may reduce severity of the symptom. Improvement might be immediate or the patient may experience gradual improvement over several weeks or months. However, caution is warranted in associating a particular health symptom with a particular health dysfunction. A health dysfunction could be ascertained and its proper function improved without a corresponding reduction in severity of the most pressing health symptom(s) of the patient. Nevertheless, improving proper function of health dysfunctions ascertained using aspects of the invention still benefits the patient and, over the course of time, may comprise one part of an overall health regimen that ultimately provides relief from the most pressing health symptom(s). Accordingly, a patient should not be allowed to pick and choose which positive findings to treat.

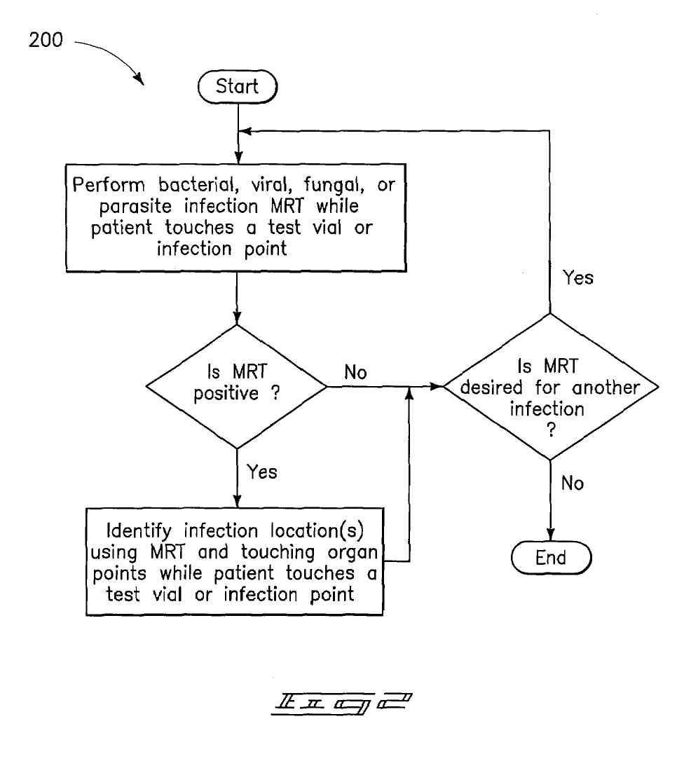

For some health dysfunctions, in the absence of complicating factors, health symptoms might be reliable indicators of a particular health dysfunction. In such circumstances, MRT to ascertain a health dysfunction could be considered optional and, instead, the dysfunction may be ascertained without MRT. In particular, the methods of FIGS. 2-10 and/or the Table below might be modified to replace MRT with a different technique. Even so, generally, MRT may be the most reliable technique to ascertain a health dysfunction.

For allergies, well-known skin scratch or blood test techniques might provide reliable indicators of a health dysfunction, namely, the allergy. Allergic reactions also might be reliable indicators when the allergen can be verified. For lactose intolerance, well-known hydrogen breath test techniques might provide reliable indicators of a health dysfunction, namely, the intolerance. Symptoms characteristic of lactose intolerance also might be reliable indicators when other dysfunctions can be ruled out as possible causes of the symptoms.

Understandably, a variety of options exist for implementing the energy medicine treatment methods according to aspects of the invention herein. For example, ascertaining a health dysfunction of a patient by using MRT includes several variations in specific techniques that are more or less effective depending upon the dysfunction being evaluated. For example, touching an organ point, stressor point, or test vial may involve the patient and/or doctor performing the touching. Similarly, various parts of the hand, namely the palm, fingertip(s), index fingertip, pad of the index finger, surface of the index fingernail, posterior surface (nail-side) of the finger(s), etc. may be used for the touching. Touching various points also includes using a magnet instead of the hand.

Also, stimulating the cranium may include tapping, massaging, or simply touching, and may further include using some device as an alternative to a finger or hand. Such other device may include a cap with numerous knobs, bumps, points, or other stimulatory extensions or may include a laser or like device. Stimulating the cranium may include running the fingertips through the hair across the scalp, patting the cranium with cupped palms, etc. Mechanical and/or other movement of the described cap and/or extensions may also sufficiently stimulate the cranium. The duration and intensity of cranial stimulation at each point on the cranium may be merely long enough for the contact to be sensed by the patient. Although, if using a laser, then the patient might not consciously sense the laser illumination of the cranium. Nevertheless, duration for the laser may be similar to what would be experienced if physically contacting the cranium. Laser intensity may be low enough to avoid tissue damage.

The process of stimulating a sufficient portion of the cranium may take as little as 10 seconds up to as long as 2 minutes, depending on doctor efficiency and the type of stimulation. That is, some doctors or techniques may take longer than others. Running fingertips through the hair across the scalp in a comb-like action may be rather quick compared to scanning with a laser, which involves more caution and time. One advantage to stimulating the cranium in the manner described may include clearing more than one head point at a time. It is conceivable that, otherwise, a doctor would locate each head point individually and clear it individually. The aspects of the invention provide a more efficient process and prevent the possibility of missing a head point.

For completeness, the “sufficiently large portion of the cranium” may include the entire surface of the cranium that directly overlies the brain. In such manner, no head point(s) is missed. However, it is conceivable that a smaller portion of the cranium may be sufficient in most cases. All of the sufficiently large portion need not be stimulated simultaneously, though it could be, especially if some device as described above is used to cover the entire cranial surface that directly overlies the brain. Conveniently, multiple passes with a finger, hand, or other device may be used to address all of the sufficiently large portion.

In another aspect of the invention, an energy medicine treatment method includes ascertaining a health dysfunction of a patient by using MRT, the dysfunction being selected from the group consisting of a fungal infection, a fungal infection in a sinus, a Candida infection, a digestive enzyme deficiency, a digestive HCl deficiency, and a scar/organ nerve energy disruption. The patient exhibits a head point(s) on a surface of the cranium, the head point(s) being biologically active in a manner conducive to treatment of the dysfunction. Without identifying the specific location of the head point(s), the method includes touching, tapping, massaging, or using some device on a sufficiently large portion of the cranium to clear the head point(s) and to enable the patient's body to improve proper function of the dysfunction. The patient's body exhibits a health stressor point corresponding to the health dysfunction, clearing the head point(s) including touching the stressor point while stimulating the cranium.

In summary, MRT to ascertain the existence of a health dysfunction generally involves using the patient's arm (deltoid), or other muscle, along with a stressor point, organ point, or test vial. MRT to ascertain an organ with which the health dysfunction is associated generally involves using the patient's arm and an organ point along with either a stressor point or test vial. Clearing the head point(s) generally involves using the patient's cranium along with a stressor point, an organ point, or both, and, optionally, a test vial instead of a stressor point. Understandably, a large number of different methods result from various combinations of who touches, how touching occurs, and what stimuli are used in ascertaining health dysfunctions, ascertaining organs associated with health dysfunctions, and clearing a head point(s). Accordingly, specific examples are given below representative of the current knowledge for most effectively applying aspects of the invention. The Table below summarizes the combinations of a stressor point, organ point, and test vial that may be used in the specific examples for testing the existence of, locating, and treating a condition. The examples that follow the Table provide additional details. Understandably, the combinations described in the Table may be altered in keeping with the various options otherwise described herein.

Methods described herein may include using MRT to determine whether a nutritional supplement is appropriate for a patient and the correct dosage. Nutritional supplement MRT may be performed by holding a plastic or glass container of a nutritional supplement against the patient's sternum while performing MRT. A positive finding (arm weakening) indicates the supplement is not appropriate for the patient, but a negative finding (arm staying strong) indicates the patient may benefit from the supplement.

Alternatively, nutritional supplement MRT may include the patient placing his index fingernail on a glass test vial containing a nutritional supplement while the doctor performs MRT. Due to the different touching technique, a positive finding (arm weakening) indicates that the patient may benefit from the supplement, but a negative finding (arm staying strong) indicates that the supplement is not appropriate for the patient. Determining the correct dose includes holding a glass or plastic container of the nutritional supplement against the patient's sternum and slowly counting from 1 to 10 while performing MRT. The patient's arm may weaken upon arrival at the correct number of tablets for the patient's daily dosage. Daily dosage may be confirmed by putting one supplement tablet at a time in the palm of the patient's hand while performing MRT. A positive finding (arm weakening) may occur upon surpassing the appropriate daily dosage by one tablet. Nutritional testing may be useful in aspects of the invention to confirm treatment effectiveness by checking the nutritional needs of an organ immediately before and after treatment.

Surrogate testing may be applicable in aspects of the invention. It involves using another person to assist in the testing process. Surrogate testing may be warranted when the patient cannot participate in MRT effectively as a result of the patient being too young or old, being too weak, having an infirmity of their upper extremity (for example, disability, amputation, etc.), using excessive strength during MRT, remaining electrically imbalanced, becoming fatigued before MRT completion, being an animal, etc. Surrogate testing an adult in the place of a child involves the child sitting on the adult's lap, allowing the child's electricity to pass through the adult. The adult's arm may be used for MRT and the adult's fingertip may be used to touch test vials. The child's stressor points and organ points may be used while using the adult's arm for MRT.

An adult acting as a surrogate for another adult includes the surrogate placing their hand on the patient's shoulder or holding the patient's hand. The surrogate's arm may be used to perform MRT while the patient touches the vials or while the doctor or patient touches the patient's stressor points or organ points. As with the patient, a surrogate may undergo initial preparations described in FIG. 1 before MRT. Also, the surrogate may be tested while not touching the patient to verify that the patient's findings exist only in the patient and, thus, are properly attributed to the patient.

When tested through a surrogate, treatment of the patient may be accomplished as described herein. When using vials, if the usual technique is problematic, then the patient may touch the vial with his fingertip or the doctor or patient may hold the vial about 1 inch below the umbilicus of an adult during treatment. For children, the distance below the umbilicus may be reduced by an amount corresponding to the child's reduced size compared to an adult.

TABLE

| Condition | Test | Locate | Treat | Ex. |
|---|---|---|---|---|
| bacteria/virus | vial or stressor | stressor/organ | stressor/organ | 1 |
| fungus | vial or stressor | stressor/organ | stressor/organ | 2 |
| (not sinus) | | or organ/vial | | |
| fungus (sinus) | vial or stressor | stressor/organ | stressor/organ | 3 |
| | | or organ/vial | | |
| fungus | vial or stressor | stressor/organ | stressor/organ/ | 4 |
| (Candida) | | or organ/vial | pulse point | |
| parasite | vial | organ/vial | organ/vial | 5 |
| parasite | stressor | NA | stressor | 6 |
| allergy | vial | organ/vial | organ/vial | 7 |
| metal/chemical | vial | organ/vial | organ/vial | 8 |
| digestive enzyme | stressor | NA | stressor | |
| scar | stressor | stressor/organ | stressor | |
| root canal | organ | stressor/organ | stressor/organ | 9 |
| organs | stressor/organ | organ | stressor/organ | 10 |
| | (2nd step) | (1st step) | | |
| organ (strengthen) | organ | organ | organ | |
| muscle | organ | organ | organ | 11 |
| subluxation | organ | organ | organ | |
| disc | organ | organ | organ | |

### Bacterial, Viral, Fungal, or Parasitic Infection

FIG. 2 shows a flow chart of a method 200 that includes performing bacterial, viral, fungal, or parasitic infection MRT while the patient touches a test vial or infection point (stressor point corresponding to a pneumococcal, Epstein-Barr viral, fungal (or Candida), staphylococcal, streptococcal, viral 2, helicobacter pylori (h. pylori), parasitic, or viral 1 infection) as indicated in FIG. 12. If using test vials, the index fingernail may be placed flush against the vial for about 1 to 2 seconds. Infection points may be touched with the fingertip.

If MRT is positive, then method 200 includes identifying an infection location(s) using MRT and touching organ points while the patient touches a test vial or infection point. Either the doctor or the patient may touch the organ points. Selecting one or the other is largely a matter of availability and efficiency. The doctor and patient each only have one hand available during MRT. Also, the doctor typically may locate organ and stressor points more quickly and step through each one. Initially, if infection MRT is positive, then the patient may remain touching the infection point while the doctor checks the organ points. As another alternative for a touching technique, the patient or doctor may hold a test vial against the organ point to determine infection location(s). In such case, the same person may touch both the test vial and the organ point simultaneously.

Treatment generally includes the patient touching the infection point while the doctor touches the organ point and stimulates a sufficiently large portion of the cranium to clear the head point(s). However, other touching scenarios are described below for the infection point and organ point. As indicated, touching the organ point is optional. Treatment effectiveness may be confirmed by performing infection MRT again using the test vial or infection point.

### Example 1

A doctor performed bacterial and viral infection MRT while a patient touched a test vial with the index fingernail or touched an infection point with the index fingertip. If MRT was positive, the doctor identified infection location(s) by using MRT and touching organ points shown in FIG. 11 while the patient touched the appropriate infection point. Treatment included the patient continuing to touch the infection point while the doctor touched the organ point corresponding to the infection location and stimulated a sufficiently large portion of the cranium to clear the head point(s) without identifying a specific location of the head point(s). Treatment effectiveness was confirmed by retesting with the infection point or test vial.

### Example 2

A doctor performed fungal (other than sinus) infection MRT while the patient touched the umbilicus with a fingertip or touched a test vial with the index fingernail. Notably, the umbilicus is the infection point for fungus. If MRT was positive, the doctor identified the infection location(s) using MRT and first touching the descending large intestine organ point shown in FIG. 11 while the patient touched the umbilicus or the test vial to rule out a Candida infection. If a Candida infection was not apparent, then the doctor continued touching other organ points. Treatment included the doctor or the patient touching the organ point corresponding to the fungus location while the patient touched the umbilicus with the fingertip and the doctor stimulated a sufficiently large portion of the cranium to clear the head point(s) without identifying a specific location of the head point(s). Treatment effectiveness was confirmed by retesting with the umbilicus or test vial.

### Example 3

When the method in Example 2 revealed an infection located in the sinuses as indicated by one of the sinus organ points shown in FIG. 11, treatment proceeded as follows. The doctor touched the involved sinus organ point(s) with his fingertip(s) while the patient touched the umbilicus with his index fingertip and the doctor lightly tapped on the top of the patient's head for 30 seconds with the fingertips of the other hand. The tapping occurred over a sufficiently large portion of the cranium to clear the head point(s) without identifying a specific location of the head point(s). Treatment effectiveness was confirmed by retesting with the test vial or umbilicus.

### Example 4

When the Example 2 method indicated a Candida infection, treatment proceeded as follows. The patient touched the umbilicus with his right index fingertip and touched the descending large intestine organ point with his left index fingertip while the doctor lightly tapped the top of the patient's head with the fingertips of his hand for no more than 2 minutes at a time. The tapping occurred over a sufficiently large portion of the cranium to clear the head point(s) without identifying a specific location of the head point(s). The day of the first treatment, the patient immediately began following an anti-Candida diet. The patient also started taking a probiotic, such as Total Probiotics available from Nutri West located in Douglas, Wyo.

Adults usually required about 4 treatments to complete Candida elimination in a safe manner. Patients sometimes felt very fatigued after the first few treatments. Children usually required 2 or 3 treatments since treatment could occur for more than 2 minutes at a time. For children or adults, treatments could be spaced about 1 day apart, for example, Monday, Wednesday, Friday, Monday, to allow for adequate detoxification between treatments. Patients sometimes experienced significant fatigue after the first couple of treatments from the die-off reaction.

Each treatment could eliminate about 30% of the Candida. Before the second treatment, the patient was asked the question, “On a scale of 1 to 10 with 10 being your starting point, how much Candida remains?” Then, the doctor counted from 1 to 10 while performing muscle testing. The patient could be at a 7 or less before the second treatment. Before the third treatment the patient could be at a 4 or less. Before the fourth treatment the patient could be at a 1 or less, that is, 90% of the Candida eliminated. Accordingly, the fourth treatment could easily complete removal of Candida. Either the Candida infection point or the test vials could test negative following the fourth treatment.

During the anti-Candida diet, patients typically were having at least two bowel movements each day. If not, the patient took a natural bowel stimulant or laxative for the first week. Alternatively, the patient used colonic irrigation. The patient drank at least eight glasses of water each day. Adults remained on the diet for two weeks after completing the fourth treatment or after testing indicated elimination of Candida. Children stayed on the diet for one week after the final treatment. The doctor recommended that the spouse of a patient or children of an infected mother undergo testing for Candida due to the possibility of infection.

### Example 5

A doctor performed parasitic infection MRT while a patient touched a test vial for about 2 seconds with the index fingernail. The doctor identified the infection location(s) using MRT while the doctor touched each organ point by holding a test vial that tested positive against it. Treatment included the patient touching the organ point by holding the test vial against it while the doctor stimulated a sufficiently large portion of the cranium to clear the head point(s) without identifying a specific location of the head point(s). After treatment, MRT was negative both with the patient holding the test vial over the organ point and with the patient lightly touching the test vial with his index fingernail. After the first treatment of an infection based upon a test vial of one type of parasite, parasitic infection MRT was repeated with test vials for other types of parasites to be certain no infection of other species was present. The doctor repeated treatment after three to four days to address the subsequently hatched generation of parasites not affected by the initial treatment, if any.

### Example 6

Parasitic infection MRT was performed while the doctor touched the parasitic infection point with his fingertip. Alternatively, the patient touched the infection point as in Examples 1 through 5. If MRT was positive, then the doctor treated the infection by touching the parasitic infection point with his fingertip while stimulating a sufficiently large portion of the cranium to clear the head point(s) without identifying a specific location of the head point(s). The doctor repeated treatment after three to four days to address the subsequently hatched generation of parasites not affected by the initial treatment, if any.

### Allergies and Metals or Chemicals

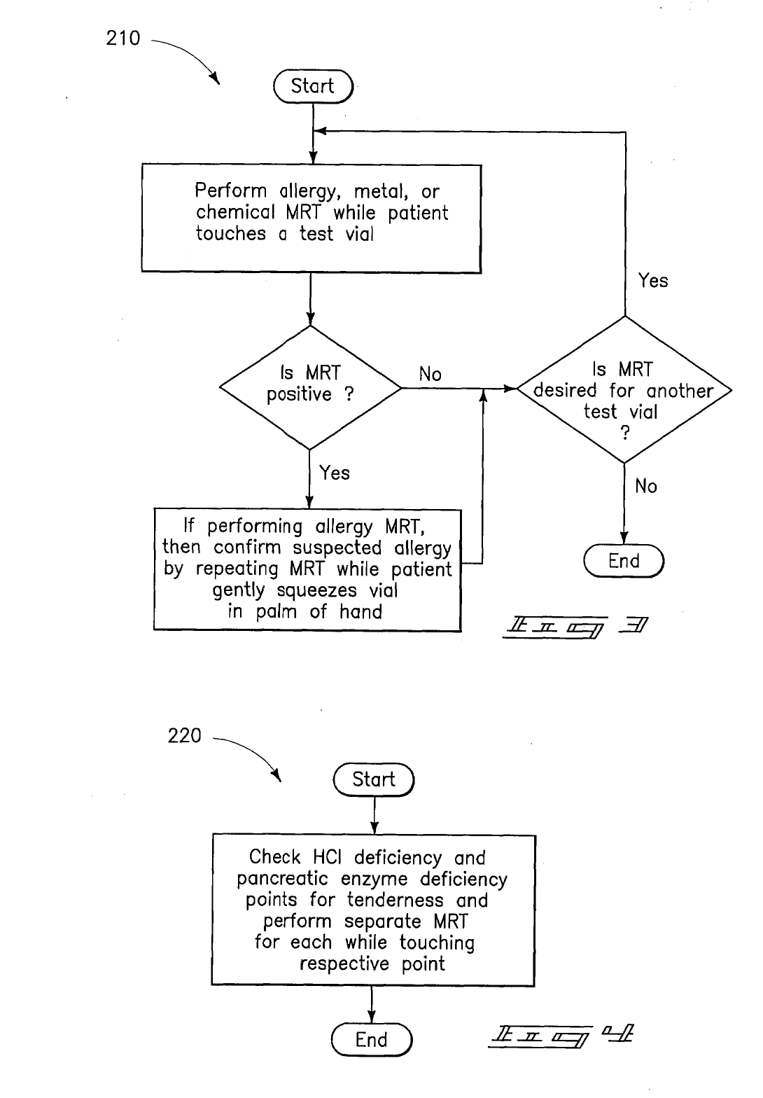

FIG. 3 shows a flow chart of a method 210 that includes performing allergy, metal, or chemical MRT while the patient touches a test vial. After all allergies in a test kit have been tested, then the doctor may confirm any suspected allergy by repeating MRT while the patient gently squeezes a corresponding vial in the palm of his hand. For convenience in confirming suspected allergies, the doctor may partially remove any suspected test vial from the kit for easy identification.

Some airborne allergies (especially seasonal) do not test positive in method 210 until other allergies are eliminated. For example, a dust allergy may cause other airborne allergies. Accordingly, method 210 may be repeated after treatment of the initially identified allergies. Organ or systemic infections may also cause allergies. For example, many environmental and food allergies result from a Candida infection. If the patient has a Candida or other infection, allergy treatment may be postponed until the Candida or other infection is eliminated. Subsequently, any allergies initially identified may be retested.

In testing for metals or chemicals, often a metal mix vial and a chemical mix vial may be used to cover a broad spectrum of metals and chemicals quickly. If a patient tests positive for a metal mix or chemical mix vial, then test vials corresponding respectively to specific metals or specific chemicals may be used to ascertain which specific metals or chemicals require treatment. In addition to or instead of metal/chemical toxicity, it is possible for a patient to be allergic to a metal or chemical. Testing for metal/chemical toxicity involves using the fingernail while testing for a metal/chemical allergy involves using the fingertip, as specified below. Testing for lactose intolerance may be conducted in the same manner as allergy testing.

Treatment of allergies and metals or chemicals may include touching the test vial while optionally touching the organ point and stimulating a sufficiently large portion of the cranium to clear the head point(s) without identifying a specific location of the head point(s). Various touching scenarios are possible for touching test vials and stressor points, as described below. Also, a substance other than a vial may be used, such as a substance including the allergen, for example, wheat, eggs, fruit, grass, dust, pet hair, etc. Further, in the field of energy medicine, known test vials are available that contain water radionically imprinted with the allergen. Such vials may be used instead of other substances, or test vials containing substances, which include the allergens themselves.

Observation indicates that treatment of lactose intolerance may include touching a test vial with the fingernail(s) and clearing head points. The middle finger nail appears to be the most effective. Interestingly, in a practice known as Korean hand acupuncture (or Koryo Soji Chim), a point called “small intestine #14” is located on the skin just slightly off of the left and right sides of the middle fingernail near where the nail cuticle meets the skin of the middle finger. Lactose intolerance is believed to be related to function of the small intestine. Similar to treatment of allergies, a substance including lactose may be used instead of a vial. For example, lactose powder may be sprinkled on the fingernail.

A doctor may exercise thoughtful clinical judgment in a treatment process for patients that exhibit serious metal or chemical toxicity or exhibit high sensitivity or reactivity to foreign substances. The patient's elimination systems are recommended to be functioning very well before treatment. Also, metals or chemicals are best treated after the patient completes any warranted Candida or other infection elimination.

### Example 7

A doctor performed allergy MRT while a patient lightly touched the center of a test vial for about 2 seconds with the tip of his index finger held at about a 45° angle to the glass side of the vial laying on its side. After obtaining a positive finding, the patient held the test vial in the palm of his hand while the doctor touched organ points with the tip of his index finger to identify the organ(s) with which the allergy was associated. Alternatively, the doctor performed MRT while holding the test vial over organ points to identify location(s) of the allergy. Treatment included the patient gently squeezing the test vial in the palm of one hand while touching the organ point of the affected organs with the fingertip(s) of his other hand and the doctor stimulating a sufficiently large portion of the cranium to clear the head point(s) without identifying a specific location of the head point(s). After treatment, the doctor performed allergy MRT again to confirm elimination of the allergy while the patient gently squeezed the vial in the palm of one hand and then the other hand. Treatment was repeated if the doctor obtained positive findings.

### Example 8

A doctor performed metals and chemicals MRT while a patient touched a metal mix test vial and then a chemical mix test vial with his index fingernail. After both test vials tested positive, the doctor performed MRT using test vials containing specific metals and chemicals while the patient touched the test vials with his index fingernail to ascertain which specific metal or chemicals required treatment. For each positive test vial, the doctor first ascertained whether the patient's liver was associated with the elevated metals or chemicals by the doctor holding each positive test vial on the patient's liver point while performing MRT.

If MRT produced a positive finding, the doctor kept the vial on the liver point and stimulated a sufficiently large portion of the cranium to clear the head point(s) without identifying a specific location of the head point(s). The doctor repeated metal or chemical MRT while the patient touched the test vial for which the patient was treated with his index fingernail. If MRT still produced a positive finding, then the doctor checked other organ points, beginning with the bladder and kidney organ points, in the same manner as indicated for the liver point. Treatment ensued for additional organ points testing positive.

### Digestive Enzymes

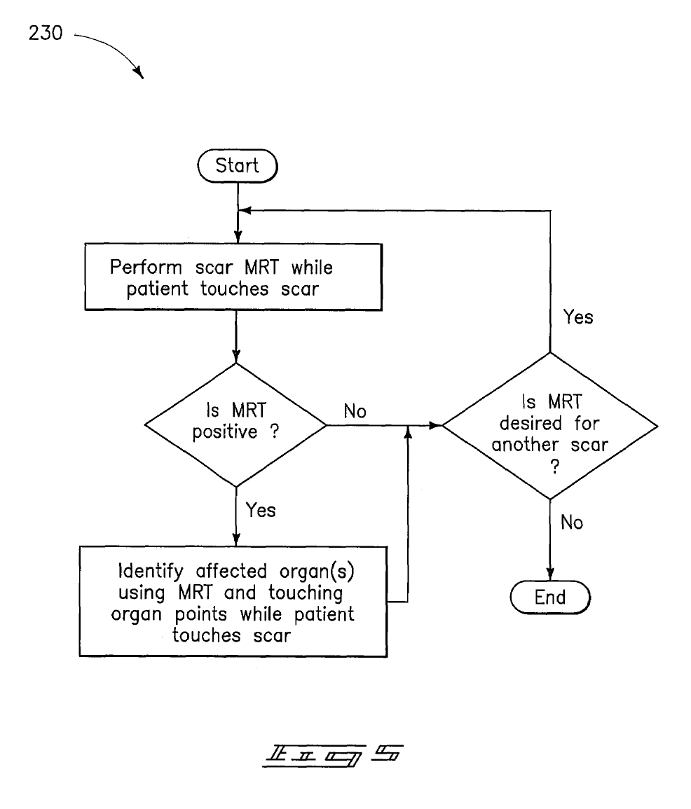

FIG. 4 shows a flow chart of method 220 that includes checking a patient's HCl deficiency and pancreatic enzyme deficiency stressor points shown in FIG. 12 for tenderness and performing MRT separately while touching the respective stressor point. The HCl deficiency and pancreatic enzyme deficiency stressor points shown in FIG. 12 are derived from the works of Robert Riddler, DC and are located about one and a half inches from the adult sternum on the lower edge of the rib cage. Treatment includes the patient holding the nail side of any finger on the respective stressor point while the doctor stimulates a sufficiently large portion of the cranium to clear the head point(s) without identifying a specific location of the head point(s).

As an additional measure, the patient may be tested before and after treatment for his need of a supplement intended to address an HCl or pancreatic enzyme deficiency. One example of a supplement addressing an HCl deficiency includes Zypan® available from Standard Process located in Palmyra, Wis. One example of a supplement addressing a pancreatic enzyme deficiency includes Multizyme® also available from Standard Process. Following treatment, the patient's dosage should be much less or zero. If the patient's dosage is not zero after treatment, then the doctor may recheck dosage after a day or more. Generally, the patient's dosage will drop to zero with the passage of a little time.

### Scars

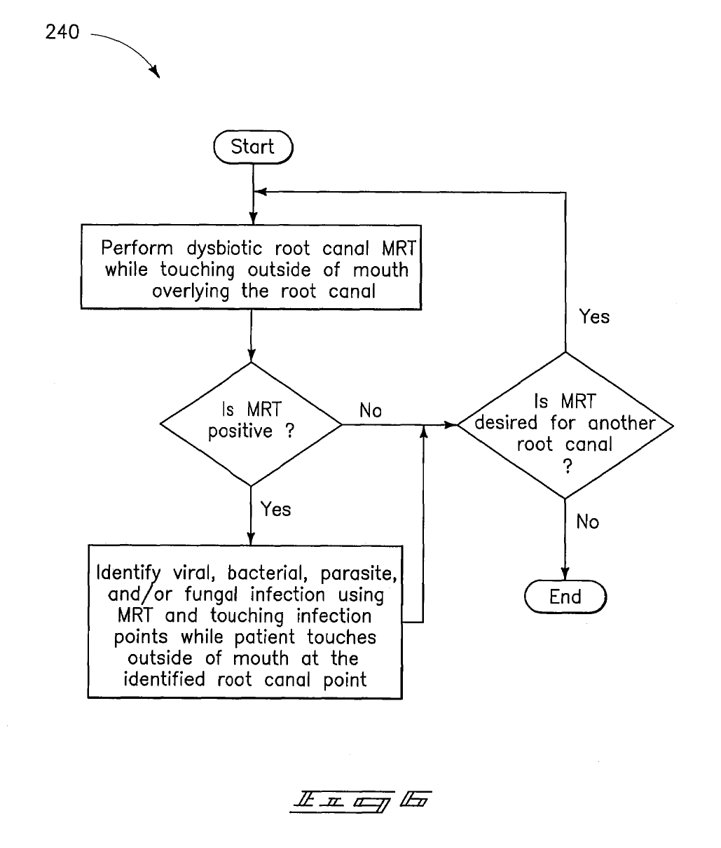

A scar may interfere with the sympathetic nervous system of a patient, causing an abnormally functioning flow of nerve energy to a specific organ. Accordingly, scars may be evaluated and treated. Essentially, the scar is considered a stressor point and is not necessarily near an affected organ. The scar may be distant, as in the example of a chin scar affecting bladder function. FIG. 5 shows a flow chart of method 230 including performing scar MRT while the patient touches a scar. After a positive finding, method 230 includes identifying affected organ(s) using MRT and touching organ points while the patient touches the scar. The patient may use his fingertip(s) to touch the scar. The method may be repeated for other scars. Treatment includes the patient touching the scar while the doctor stimulates a sufficiently large portion of the cranium to clear the head point(s) without identifying a specific location of the head point(s). The patient may optionally touch the organ point during treatment. After treatment, scars and scar/organ connections may be retested.

### Dysbiotic Root Canals

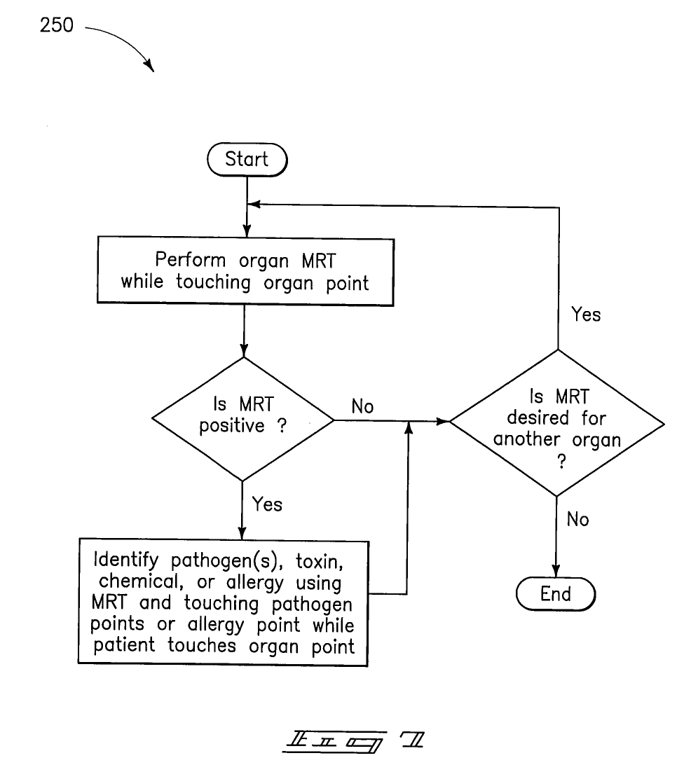

Since almost any part of the body may be negatively impacted by a viral, bacterial, parasitic, and/or fungal infection, a nerve root of a tooth, as a result of a root canal procedure, may become infected with such pathogen(s). Surprisingly, elimination of the nerve root infection may be a key to addressing a patient's health symptoms. Accordingly, FIG. 6 shows method 240 that includes performing dysbiotic root canal MRT while touching the outside of the patient's mouth overlying the root canal. After a positive finding, method 240 includes identifying a viral, bacterial, parasitic, and/or fungal infection using MRT and touching the specific infection points shown in FIG. 12 while the patient touches the outside of his mouth at the identified root canal point.

Treatment of an acutely infected nerve root may remove the infection and relieve pain associated with the infection. The location is identified by its associated pain and treatment may occur by the patient touching the infection location while the doctor clears the head point(s). It is recommended that the doctor also touch the corresponding infection point during treatment. Since the root is decaying, the infection may return, usually within a few days. Accordingly, the risk of a returning infection may warrant the patient subsequently obtaining a root canal procedure.

Essentially, a root canal infection is ascertained and treated in a manner analogous to other bacterial, viral, fungal, and parasitic infections wherein the nerve root is the organ associated with the infection. However, dysbiotic root canals may be most effectively addressed merely by inquiring with a patient whether the patient received a root canal procedure instead of checking each nerve root as one of the organ points, such as those in FIG. 11. Observation indicates that dysbiotic root canals rarely produce positive tests for bacterial, viral, fungal, and parasitic infections using infection (stressor) points or vials. It is believed this may be due to the relatively small blood supply to the area. Notably, the socket remaining after a tooth extraction may be infected like a root canal and may be tested and treated in the same manner as described.

### Example 9

A doctor inquired with a patient and ascertained that the patient previously underwent a root canal procedure. The doctor performed dysbiotic root canal MRT while touching the outside of the mouth with the tip of the doctor's index finger. The doctor also touched nearby points along the patient's gum line outside the mouth. After a positive finding, the patient held the tip of his index finger on the identified point while the doctor checked viral, bacterial, parasitic, and/or fungal infection points using MRT until obtaining a positive finding. Treatment included the patient continuing to touch the infection location (optional) while the doctor touched the infection point with the tip of his index finger and stimulated a sufficiently large portion of the cranium to clear the head point(s) without identifying a specific location of the head point(s). Treatment effectiveness was confirmed by retesting the infection location.

### Organs

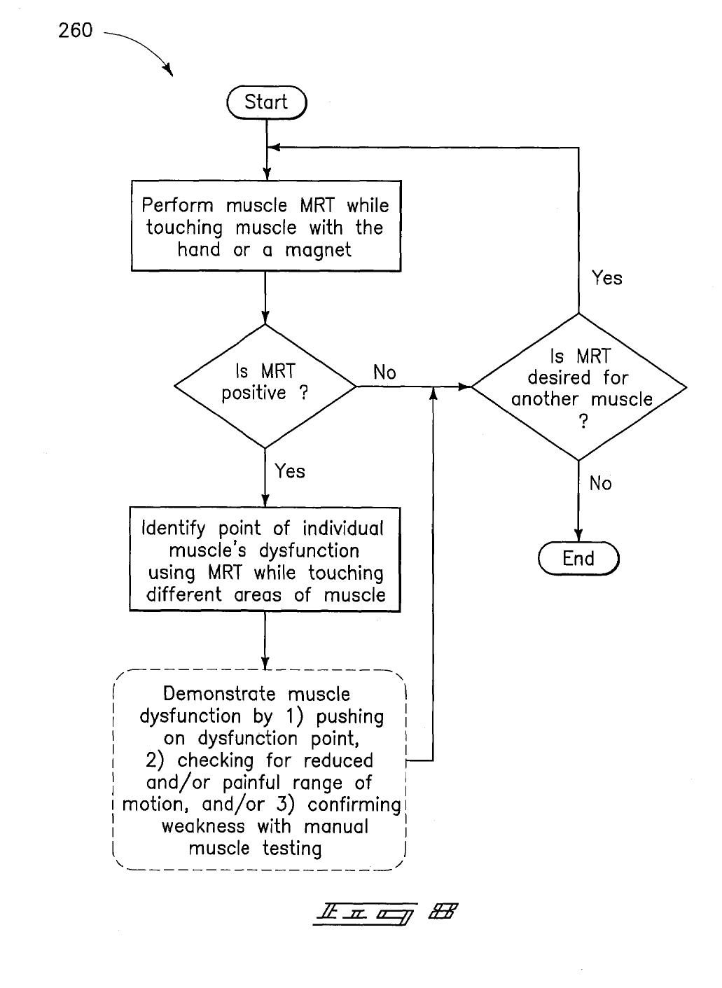

Methods described above involve using health stressors or test vials to ascertain an infection, allergy, elevated metals or chemicals, digestive deficiency, or nerve energy disruption. After ascertaining the problem, the location of the problem could be identified, if desired. As an alternative, ascertaining a health dysfunction may instead include first identifying the location of a problem followed by ascertaining the specific type of dysfunction. Accordingly, FIG. 7 shows a flow chart of method 250 including performing organ MRT while touching an organ point. In the case of the hypothalamus organ point, the patient may touch it with his tongue instead of the doctor using a fingertip or fingernail inside the patient's mouth for testing or treatment.

After a positive finding, a specific pathogen, allergy, toxin, or chemical (drug) dysfunction may be identified using MRT and touching pathogen, allergy, toxin, or chemical stressor points while the patient touches the organ point. The pathogen, allergy, toxin, and chemical stressor points are shown in FIG. 12. After a dysfunction is identified, it may be treated according to the methods described herein. The toxin stressor is used to address toxic substances, i.e. metals or chemicals, to which the patient was exposed. The chemical stressor is used to address intentionally administered drugs that accumulated in the body to the point that they give an adverse effect. Such drugs are typically chemicals.

Warts, cysts, and other infections that are encapsulated and/or highly localized generally do not test positive merely using stressor points. However, if discovered (perhaps by another means), they may be tested or treated by touching the localized infection during testing or treatment as if it were an organ point.

Potentially, an organ may produce a positive finding but no pathogen, allergy, toxin, or chemical can be identified. Such a dysfunction may result from an organ being weakened by chronic stress, such as the thyroid, adrenals, etc. After ruling out pathogen, allergy, toxin, and chemical stressor points, the doctor may perform organ MRT while touching the organ point with the nail side of his fingers. A positive finding indicates that the organ may be amenable to treatment to strengthen the organ without using nutritional supplements.

Treatment of the weak organ includes the doctor placing the nail side of his fingers over the organ point while stimulating a sufficiently large portion of the cranium to clear the head point(s) without identifying a specific location of the head point(s). Before treatment, the doctor may select a nutritional supplement appropriate for the affected organ and determine a daily dosage. After treatment, dosage for the nutritional supplement may be checked again to confirm treatment effectiveness. Generally, after strengthening an organ or treating its infection, allergy, toxin, or chemical, the nutritional requirements of the organ may be significantly reduced.

### Example 10

A doctor performed organ MRT while touching organ points with his fingertip(s), except for the heart point, which was tested with the palm of his hand, and the four sections of the large intestine, which were tested with the little finger edge of his hand. The thumb edge of the hand could be used with the thumb tucked under. After a positive finding, the doctor touched the following stressor points in the order indicated, which reflects the order most frequently found with the most common being first: virus 1, virus 2, streptococcus, h. pylori (small intestine point), allergy, parasite, fungus, toxin, staphylococcus, chemical, and pneumococcus. Any infection, allergy, toxin, or chemical identified was treated according to methods described herein.

### Muscle Dysfunction

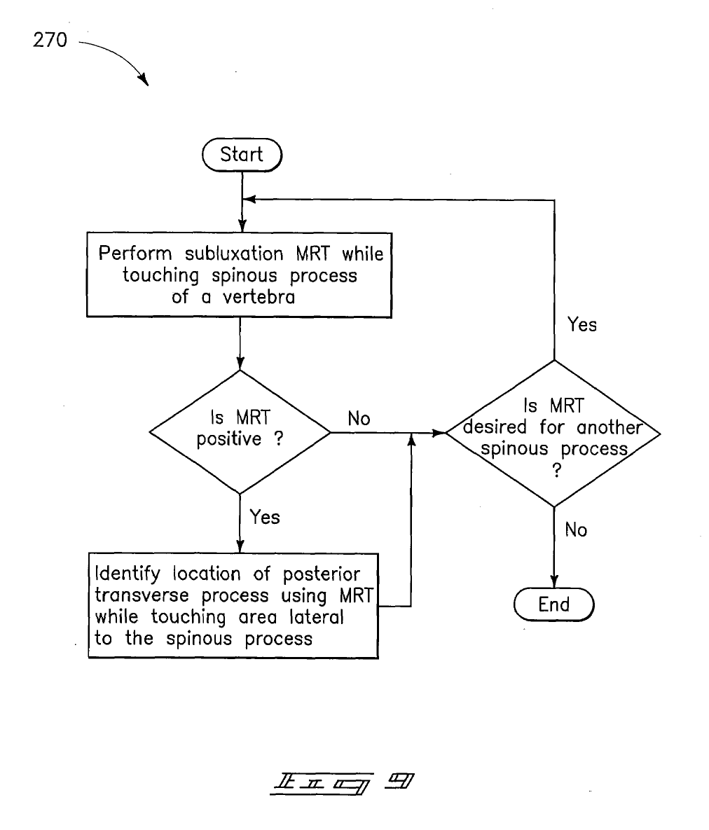

FIG. 8 shows a flow chart of method 260 including performing muscle MRT while touching a muscle with the hand or a magnet. After a positive finding, points of an individual muscle's dysfunction are identified using MRT while touching different points of the muscle. As an optional step, depending upon whether the patient previously noticed a muscle dysfunction, the now apparent dysfunction may be demonstrated by 1) pushing on the dysfunction point to reveal any muscle tenderness, 2) checking for a reduced and/or painful range of motion, and/or 3) confirming weakness with manual muscle testing. When evaluating a specific area of the patient's body, for example, the knee, the doctor may test all muscles that could possibly influence the function of the specific area.

Treatment includes the doctor touching the dysfunction point and stimulating a sufficiently large portion of the cranium to clear the head point(s) without identifying a specific location of the head point(s). Treatment effectiveness may be confirmed by repeating manual muscle testing to show improved strength and/or checking for increased and/or less painful range of motion. Pushing on the dysfunction point might reveal that the muscle belly is much less tender. However, even if the muscle is only a little less tender, the remaining muscle tenderness does not determine treatment success and, instead, depends upon the amount of lactic acid accumulation.

With regard to the part of the hand or magnet used in the touching, distinction may be made between hypertonic and hypotonic muscles. Hypertonic refers to muscles having more than normal tone or, in other words, being in a state of contraction. Hypotonic refers to muscles having less than normal tone or, in other words, being in a state of expansion. MRT may be used for identifying whether a muscle is hypertonic or hypotonic. With the patient in a relaxed state, a hypertonic muscle may weaken using the posterior surface (nail side) of the hand or the north pole of a magnet. A hypotonic muscle may weaken using the anterior surface (palm side) of the hand or the south pole of a magnet.

For treating hypertonic muscle, the nail side of the hand or the north pole of a magnet may be used. For treating hypotonic muscle, the palm side of the hand or the south pole of a magnet may be used. Without being limited to any particular theory, it is believed that, since hypertonic muscle exhibits a positive electromagnetic field, the north (negative) pole of the magnet used while stimulating head points cancels out the positive field of hypertonic muscle and normalizes the muscle. The nail side of the handle may function similarly. A corresponding function is believed to exist for the south pole of a magnet and palm side of the hand in relation to hypotonic muscle. Muscle often works in pairs where one muscle contracts, while another expands, to move a body part one direction. The one muscle expands, while the other muscle contracts, to reverse the direction of movement. The bicep/tricep pair is an appropriate example.

Observation indicates that either muscle in a pair may be treated to address a dysfunction in one or the other muscle. For example, treatment of a hypertonic muscle with the nail side of the hand or the north pole of the magnet treats both the hypertonic muscle and the hypotonic muscle in a pair to produce a balancing effect. Vice versa works as well. Recognition of such technique may be especially useful when one muscle is more accessible than the other.

### Example 11

A doctor performed muscle MRT while touching a muscle area to be evaluated with the posterior surface (nail side) of one or more fingers, depending upon the size of the muscle area, or using the north pole of a magnet. Specifically, touching was accomplished by the doctor slowly sliding his finger(s) or the magnet over the surface of a specific muscle during MRT until a positive finding occurred. Next, the doctor isolated the problem area, usually the belly of the muscle, with the nail side of the finger(s) or the magnet during MRT. Muscle dysfunction was demonstrated by revealing muscle tenderness, reduced and/or painful range of motion, and/or weakness during manual muscle testing. Muscle dysfunction was treated by the doctor holding the nail side of his finger(s) or the north pole of the magnet over the dysfunction point and stimulating a sufficiently large portion of the cranium to clear the head point(s) without identifying a specific location of the head point(s).

### Subluxations

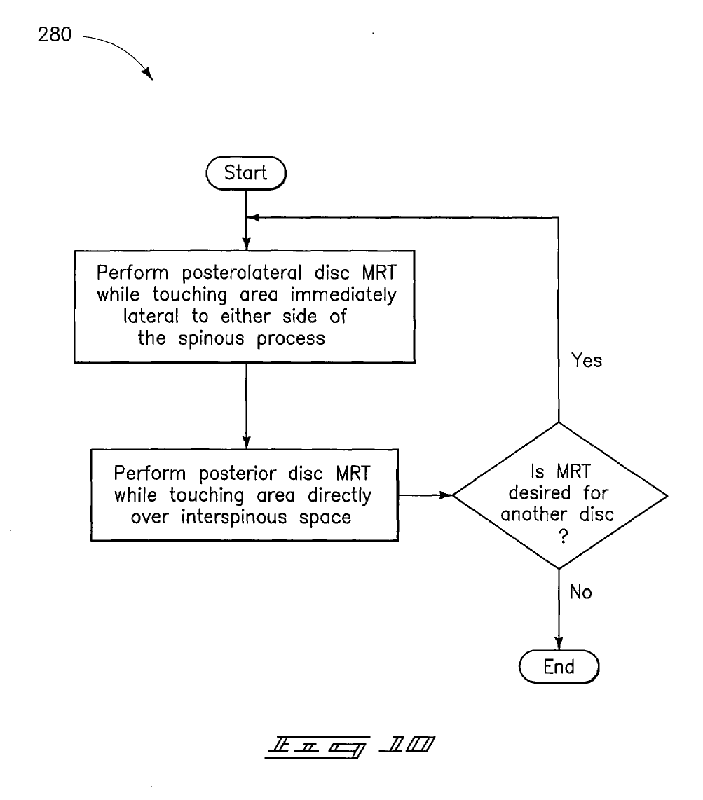

FIG. 9 shows a flow chart of method 270 that includes performing subluxation MRT while touching the spinous process of a vertebra. As noted above for allergies, lactose intolerance, and other health dysfunctions, known reliable indicators of a subluxation may be used instead. One indicator may include identifying a painful range of motion. After a positive finding, the location of the posterior transverse process (that is, the transverse process out of alignment in the direction of the patient's posterior) is identified using MRT while touching the area immediately lateral to the spinous process. The doctor may touch the spinous process with his fingertip(s) to identify a positive vertebra and may use the nail side of his finger while locating the posterior transverse process. Bones, joints, or vertebrae that are anterior may be tested as above.

Treatment includes touching the posterior transverse process while stimulating a sufficiently large portion of the cranium to clear the head point(s) without identifying a specific location of the head point(s). The doctor may use the nail side of any finger during treatment. Anterior bones, joints, or vertebrae may be treated using the palm of the hand or pad of the finger(s). Alternatively, if possible, a subluxated bone, joint, or vertebra may be oriented in a position that makes the subluxation slightly worse (to call attention to a particular bone, joint, or vertebra) while stimulating a sufficiently large portion of the cranium to clear the head point(s) without identifying a specific location of the head point(s).

If related nerve root compression is causing muscle weakness, the doctor may check the appropriate muscle(s) before and after treatment to verify effectiveness. If treatment of the subluxation does not improve muscle strength, the doctor may test (manually) and treat appropriate discs or muscles.

### Spinal Discs

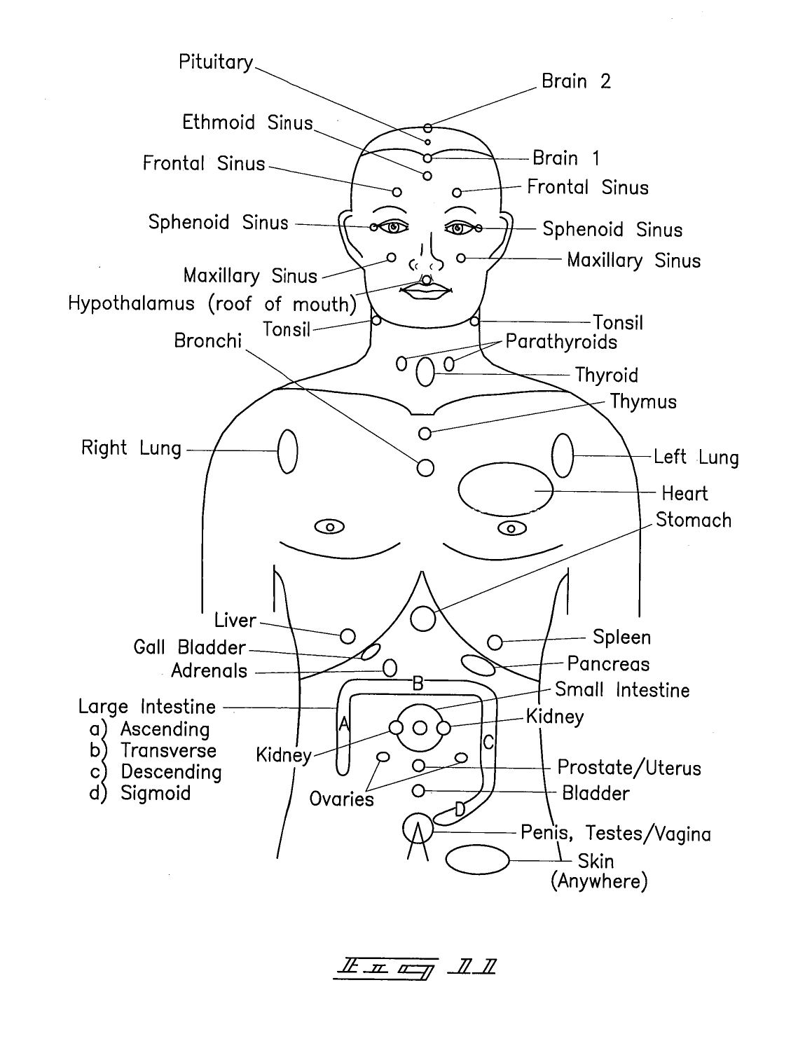

FIG. 10 shows a flow chart of method 280 that includes performing postero-lateral disc MRT while touching the area immediately lateral to either side of the spinous process. A “postero-lateral” disc refers to a disc out of alignment in the lateral direction of the patient's posterior. In method 280, posterior disc MRT is next performed while touching the area directly over the interspinous space. A “posterior” disc refers to a disc out of alignment in the direction toward the patient's posterior. The doctor may use the nail portion of his index finger for MRT.

Before treating a disc, the doctor may first treat any subluxated vertebra and release any tight muscles in the area. Treatment includes the doctor holding the index fingernail flush against the point that produced a positive finding while stimulating a sufficiently large portion of the cranium to clear the head point(s) without identifying a specific location of the head point(s). Alternatively, the patient may be oriented into the position that makes the disc pain the worst (to call attention to a particular disc) while stimulating a sufficiently large portion of the cranium to clear the head point(s) without identifying a specific location of the head point(s). A disc usually takes more than one treatment. However, progressive subjective and objective improvement may occur with each treatment. After the alternative treatment, the patient may first return to a neutral position and then the process repeated. Disc treatment may continue until MRT no longer produces a positive finding. At that point, patient symptoms may be eliminated unless subluxations or muscle spasms that need releasing (piriformis, etc.) become apparent.

The patient may be treated multiple times in a day during the time period that MRT produces a positive finding. The patient may be retested the day after MRT stopped producing a positive finding to make sure the disc remained corrected. Treatment effectiveness may be confirmed by checking the patient's range of motion and/or muscle strength before and after each treatment. Also, the patient may rate his pain on a scale of 1 to 10 before and after treatment.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

I claim:

1. An energy medicine treatment method comprising:
ascertaining a health dysfunction of a patient, the patient exhibiting a head point on a surface of the cranium and the head point being biologically active in a manner conducive to treatment of the dysfunction; and
without identifying a specific location of the head point, stimulating a sufficiently large portion of the cranium to clear the head point and to enable the patient's body to improve proper function of the dysfunction.

2. The method of claim 1 wherein ascertaining the health dysfunction comprises using allergy skin scratch or blood test techniques, checking for symptoms characteristic of allergic reactions, using lactose intolerance hydrogen breath test techniques, or checking for symptoms characteristic of lactose intolerance.

3. The method of claim 1 wherein a health symptom of the patient appears to be caused by the health dysfunction and clearing the head point reduces severity of the symptom.

4. The method of claim 1 wherein clearing the head point comprises clearing multiple head points simultaneously.

5. The method of claim 1 wherein stimulating the cranium comprises touching, tapping, massaging, or using some device.

6. The method of claim 1 wherein the health dysfunction is selected from the group consisting of a fungal infection, a fungal infection in a sinus, a Candida infection, elevated metals or chemicals, a digestive enzyme deficiency, a digestive HCl deficiency, a scar/organ nerve energy disruption, muscle dysfunction, vertebral subluxation, and a posterior or postero-lateral spinal disc.

7. The method of claim 1 wherein the patient's body exhibits a health stressor point corresponding to the health dysfunction and clearing the head point comprises touching the stressor point while stimulating the cranium.

8. The method of claim 7 wherein the health dysfunction comprises a bacterial, viral, fungal, or parasitic infection, a digestive enzyme deficiency, a digestive HCl deficiency, a scar/organ nerve energy disruption, a dysbiotic root canal, or organ dysfunction.

9. The method of claim 7 further comprising ascertaining an organ with which the health dysfunction is associated by using by using MRT, the patient's body exhibiting an organ point and clearing the head point comprising touching the organ point while touching the stressor point and stimulating the cranium.

10. The method of claim 9 wherein the health dysfunction comprises a bacterial, viral, fungal, or parasitic infection, a dysbiotic root canal, or organ dysfunction.

11. The method of claim 1 further comprising ascertaining an organ with which the health dysfunction is associated by using MRT, the patient's body exhibiting an organ point and clearing the head point comprising touching the organ point while stimulating the cranium.

12. The method of claim 1 wherein ascertaining the health dysfunction comprises touching a test vial during MRT.

13. The method of claim 12 further comprising ascertaining an organ with which the health dysfunction is associated by using MRT, the patient's body exhibiting an organ point corresponding to the organ and ascertaining the organ comprising touching the organ point while touching the test vial during MRT.

14. The method of claim 13 wherein touching the organ point while touching the test vial comprises holding the test vial against the organ point.

15. An energy medicine treatment method comprising:
ascertaining a health dysfunction of a patient, the dysfunction including lactose intolerance or an allergy, the patient exhibiting a head point on a surface of the cranium, and the head point being biologically active in a manner conducive to treatment of the dysfunction; and
without identifying a specific location of the head point, stimulating a sufficiently large portion of the cranium, while the patient touches a test vial or a substance including lactose or including an allergen corresponding with the allergy, to clear the head point and to enable the patient's body to improve proper function of the dysfunction.

16. The method of claim 15 wherein ascertaining the health dysfunction comprises using allergy skin scratch or blood test techniques, checking for symptoms characteristic of allergic reactions, using lactose intolerance hydrogen breath test techniques, or checking for symptoms characteristic of lactose intolerance.

17. An energy medicine treatment method comprising:
ascertaining a health dysfunction of a patient, the dysfunction including a muscle dysfunction, the patient exhibiting a head point on a surface of the cranium and the head point being biologically active in a manner conducive to treatment of the dysfunction; and
without identifying a specific location of the head point, touching a muscle corresponding with the dysfunction while stimulating a sufficiently large portion of the cranium to clear the head point and to enable the patient's body to improve proper function of the dysfunction.

18. The method of claim 17 wherein the corresponding muscle exhibits a dysfunction point or the corresponding muscle is a hypertonic or hypotonic muscle paired with another muscle exhibiting a dysfunction point.

19. The method of claim 17 wherein touching the muscle comprises using a nail side of a hand or a north pole of a magnet for a hypertonic muscle or using a palm side of the hand or a south pole of the magnet for a hypotonic muscle.

20. An energy medicine treatment method comprising:
ascertaining a health dysfunction of a patient by using muscle response testing (MRT), the dysfunction being selected from the group consisting of a fungal infection, a fungal infection in a sinus, a Candida infection, a digestive enzyme deficiency, a digestive HCl deficiency, and a scar/organ nerve energy disruption, the patient exhibiting a head point on a surface of the cranium, and the head point being biologically active in a manner conducive to treatment of the dysfunction; and
without identifying a specific location of the head point, touching, tapping, massaging, or using some device on a sufficiently large portion of the cranium to clear the head point and to enable the patient's body to improve proper function of the dysfunction, the patient's body exhibiting a health stressor point corresponding to the health dysfunction and clearing the head point including touching the stressor point while stimulating the cranium.
